(19) United States
(12) Patent Application Publication
(10) Pub. No.: US 2020/0061931 A1
(43) Pub. Date: Feb. 27, 2020

(54) METHOD OF WELDING A PROTECTIVE STRUCTURE TO AN OPTICAL FIBER TIP

(71) Applicant: Optical Integrity, Inc., Panama City Beach, FL (US)

(72) Inventors: Joe D. Brown, Panama City Beach, FL (US); Daniel Malphurs, Panama City Beach, FL (US)

(73) Assignee: OPTICAL INTEGRITY, INC.

(21) Appl. No.: 16/546,883

(22) Filed: Aug. 21, 2019

Related U.S. Application Data
(60) Provisional application No. 62/720,331, filed on Aug. 21, 2018.

(51) Int. Cl. B29C 65/16; B29C 65/00; A61B 18/22

(52) U.S. Cl. CPC B29C 65/1635; B29C 66/7465; B29K 2709/08; B29C 66/912; A61B 18/22; B29C 66/53

(57) ABSTRACT

A method of welding a protective structure to a tip of a surgical laser fiber involves inserting the optical fiber into a length of tubing, and positioning the fiber and the tubing in a rotatable fixture arranged to simultaneously rotate the fiber and tubing while laser radiation is directed through a transparent material of the tubing, so that one or both contacting surfaces of the tubing and the fiber absorb the laser radiation and heat up to weld the tubing to the fiber. The tubing may be a length of polymer material.

This application claims the benefit of U.S. Provisional Patent Application Ser. No. 62/720,331, filed Aug. 21, 2019, and incorporated herein by reference.

### BACKGROUND OF THE INVENTION

### 1. Field of the Invention

The invention relates to the field of laser surgery, and in particular to a method of attaching a fiber tip protective structure or standoff tip to a surgical laser fiber by welding the fiber tip protective structure to the fiber.

### 1. DESCRIPTION OF RELATED ART

The method of the invention may be applied, by way of example and not limitation, to fiber tip protective structures such as the standoff soft tips described in the inventor's PCT Publication No. WO 2017/192869 and Provisional Patent Appl. Ser. No. 16/353,225, filed Mar. 14, 2019; each of which discloses standoff soft tips that serve to maintain a minimum distance between the distal end of the fiber and a stone during a laser lithotripsy procedure. Such standoff soft tips offer numerous advantages, including reduced fiber wear or erosion, protection of the interior surface of the scope through which the tip is inserted, and even facilitation of a Moses effect that reduces attenuation of the treatment laser and therefore allows use of a lower power laser and/or reduces treatment times.

A problem with fiber tip protective structures is that it is difficult to safely and economically secure the protective structures to the fiber. Conventional methods involve use of adhesives, crimping, or heat shrinking of the standoff tip, with or without an external crimp or heat shrink sleeve. However, these methods all have disadvantages. The disadvantages may include, depending on the method, high cost and low yield, or the presence of gaps, optical discontinuities, or thermal mismatches at the interface between the tip and the fiber. One potential solution, which is known to be generally safe and economical, is welding. However, welding has previously been disregarded as an attachment method for fiber tip protective structures because it is not apparent how one would achieve a weld when the interface to be welded is within the protective structure.

U.S. Pat. No. 6,282,349 (Griffin) includes a detailed discussion of the disadvantages of using adhesives to attach a quartz ferrule to a fiber, including contamination by the outgassing adhesive of the laser output lens and the possibility of sudden, explosive failure of the fiber termination as well as low yield if the viscosity of the adhesive is increased to avoid outgassing or the ferrule is countersunk to move the adhesive away from the end of the fiber. In addition, the Griffin patent discusses the disadvantages of crimping the ferrule directly to the fiber, including lowered connector mass and incompatibility with surgical laser interlocks. Griffin's solution is to insert the ferrule into a cylindrical beam block having an extension of the polymer-clad buffer portion of the fiber extending outside the ferrule, and to crimp the extension onto the fiber.

The present invention, in contrast, not only does not require adhesives, but also does not require any additional structures, including heat sleeves or beam blocks of the type disclosed by Griffin. Instead, the present invention enables welding of the protective structure directly to the fiber tip, without any additional cost-increasing and yield-lowering manufacturing steps.

### SUMMARY OF THE INVENTION

It is an objective of the invention to provide a way to attach a protective structure or standoff tip to a surgical laser fiber over an area that extends around the circumference of the fiber, without gaps or discontinuities, resulting in a product that is safe and economical to produce.

It is a further objective of the invention to provide a method of welding a protective structure or standoff tip to a surgical laser fiber.

The method of the invention may be applied to any standoff or protective tip configuration, including those described in the inventor's PCT Publication No. WO 2017/192869 and Provisional Patent Appl. Ser. No. 62/648,108.

In a preferred embodiment of the invention, a length of polymer tubing that will form the standoff or fiber tip protective structure is positioned relative to the section of fiber to which the tubing is to be attached. The polymer tubing is made of a material having high transmissivity to laser radiation. One or both of the respective surfaces of the tubing and the fiber at the interface between the tubing and the fiber is made of a material that absorbs radiation and therefore heats up when laser radiation is directed through the tubing to weld the tubing to the fiber.

A circumferential weld is achieved by positioning the fiber and polymer tubing in a lathe or similar rotating machine, and holding the fiber and tubing in contact synchronously rotating the tubing and the fiber and applying the weld energy through the polymer tubing. Heating of the materials is monitored by detecting changes in transmission and reflection of ambient lighting resulting from heat-induced changes in the optical properties of the materials to which the welding energy is applied.

Although the tubing of the illustrated embodiment is described as a length of polymer tubing, which forms a soft tip protective structure, it is within the scope of the invention to utilize tubing or cylindrical structures that are made of materials other than polymers, so long as a portion of the structure can be made transparent to laser radiation and, preferably, will absorb heat so that it can be fused to the fiber.

### BRIEF DESCRIPTION OF THE DRAWINGS

FIG. 1 is a side view of a standoff tip undergoing a welding procedure in accordance with the principles of a preferred embodiment of the invention.

### DETAILED DESCRIPTION OF THE PREFERRED EMBODIMENTS

Throughout the following description and drawings, like reference numbers/characters refer to like elements. It should be understood that, although specific exemplary embodiments are discussed herein there is no intent to limit the scope of present invention to such embodiments. To the contrary, it should be understood that the exemplary embodiments discussed herein are for illustrative purposes, and that modified and alternative embodiments may be implemented without departing from the scope of the present invention.

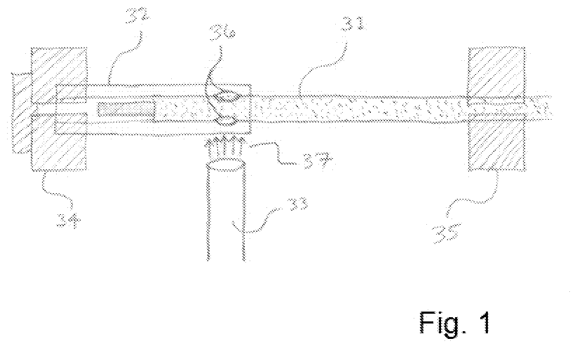

As illustrated in FIG. 1, a polymer layer 39 is stripped or removed from an end section 38 of fiber 31 to a distance that is commonly used in the industry (for example 5 mm) leaving an underlying cladding layer made of a hard polymer material such as fluoroacrylate and the glass fiber core intact. A length of radiation-transmissive polymer tubing 32 that forms a standoff tip of the type disclosed in copending PCT Publication No. WO 2017/192869 or Provisional Patent Appl. Ser. No. 62/648,108 is positioned over the stripped end section 38 and a remaining section 39 coated with a pigmented and laser absorptive polymer fiber coating. The tubing 32 and fiber 31 are held by respective fixtures 34 and 35 that permit the tubing 32 and fiber 31 to be simultaneously rotated. Fixtures 34 and 35 may, for example, consist of or include the jaws, collets, or compression fittings of a lathe or similar rotating machine, and are rotated at the same speed to prevent distortion or twisting of the tubing 32 relative to the fiber 31 during welding.

During welding, a waveguide 33 is positioned so that a laser beam 37 transmitted through the waveguide will irradiate a section of the overlapped polymer materials. The laser is then activated to heat the area 36 to be welded as the tubing and fiber are rotated. Alternatively, the laser beam may be directed at the weld point by a movable mirror, or by a lens system, or by physically moving the laser source or the parts to be welded so that the beam may be properly positioned along an axis of the fiber and tubing during welding.

Heating of the materials is monitored by monitoring transmission and reflection of ambient lighting. As the materials are heated, their optical properties change. The resulting thermal lensing effect allows the welding operator or optical sensors to visualize the temperature of the melting materials. When the materials reach a temperature sufficient to allow fusion, the laser is deactivated to enable the parts to cool. The parts may then be inspected under various lighting conditions to verify a complete weld.

Although a specific protective structure is illustrated, the fiber tip protective structures to which the method of the invention may be applied include caps, ferrules, sleeves, sheaths, standoff catheters, or any other protective structures that are placed over and fixed to the distal or treatment end a surgical laser optical fiber, for purposes that are not limited to protection of the fiber tip or scope or to any particular type of surgical applications. Accordingly, the length of tubing that forms the protective structure is not limited to a particular material, and in particular is not limited to polymers.

What is claimed is:

1. A method of attaching a protective structure to a tip of an optical fiber, comprising the steps of:
positioning, in a rotatable fixture, the fiber and a length of tubing configured to serve as the protective structure; and
simultaneously rotating the fiber and length of tubing while directing laser radiation through a transparent material of the length of tubing,
wherein one or both of respective contacting surfaces of the tubing and the fiber is made of a material that absorbs the laser radiation and therefore heats up when the laser radiation is directed through the transparent material of the tubing to weld the tubing to the fiber and create a weld or fused area that extends circumferentially around the fiber.

2. The method of attaching a protective structure to a tip of an optical fiber as recited in claim 1, further comprising the step of monitoring heating by detecting changes in transmission and reflection of ambient lighting resulting from heat-induced changes in the optical properties of the material or materials to which the welding energy is applied.

3. The method of attaching a protective structure to a tip of an optical fiber as recited in claim 1, wherein the tubing is a length of polymer tubing.

4. Apparatus for attaching a protective structure to a tip of an optical fiber, comprising:
a rotatable fixture arranged to relatively position and rotating an optical fiber and a length of tubing into which the optical fiber has been inserted; and
a waveguide for directing laser radiation through a transparent material of the length of tubing so that one or both of respective contacting surfaces of the tubing and the fiber is made of a material that absorbs the laser radiation and therefore heats up when the laser radiation is directed through the transparent material of the tubing to weld the tubing to the fiber and create a weld or fused area that extends circumferentially around the fiber.

5. Apparatus as claimed in claim 4, wherein the tubing is a length of polymer tubing.
